(19) United States
(12) Patent Application Publication
(10) Pub. No.: US 2012/0130110 A1
(43) Pub. Date: May 24, 2012

(54) PROCESS FOR CONTINUOUSLY PREPARING DIALKYL CARBONATE

(75) Inventors: Pieter Ooms, Krefeld (DE); Friedhelm Risse, Koln (DE); Andre Düx, Bornheim (DE); Carsten Buchaly, Dusseldorf (DE); Thomas Pancur, Altenholz (DE); Arthur Susanto, Koln (DE); Georg Ronge, Dusseldorf (DE); Johan Vanden Eynde, Zwijnaarde (BE); Wim Wuytack, Zele (BE)

(73) Assignee: Bayer MaterialScience AG, Leverkusen (DE)

(21) Appl. No.: 13/280,423

(22) Filed: Oct. 25, 2011

(30) Foreign Application Priority Data
Oct. 26, 2010 (DE) 10 2010 042 936.8

(51) Int. Cl. C07C 68/08

(52) U.S. Cl. 558/277

(57) ABSTRACT

The present invention relates to a process comprising purifying dialkyl carbonates of the formula (II) in one or more columns in the presence of an alkylene oxide of the formula (V) and of an alkyl alcohol of the formula (IV)

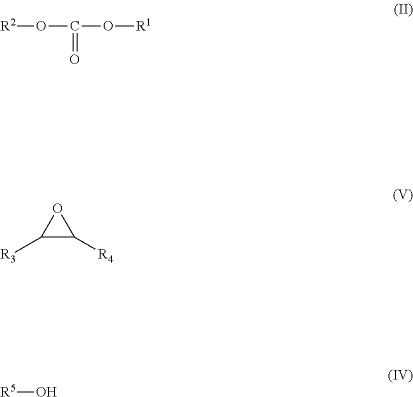

wherein R¹ and R² represent, independently of one another, linear or branched, substituted or unsubstituted C₁-C₆-alkyl, R³ and R⁴ represent, independently of one another, hydrogen, substituted or unsubstituted C₁-C₄-alkyl, substituted or unsubstituted C₂-C₄-alkenyl or substituted or unsubstituted C₆-C₁₂-aryl, and R⁵ represents a straight-chain or branched C₁-C₄-alkyl; and wherein the mean residence time of the dialkyl carbonate in the column(s) is from 0.3 h to 3 h.

### CROSS REFERENCE TO RELATED APPLICATIONS

Priority is claimed to German patent Application No. 10 2010 042 936.8, filed Oct. 26, 2010, which is incorporated herein by reference in its entirety for all its useful purposes.

### BACKGROUND

The field of the present invention relates to a continuous process for purifying a dialkyl carbonate/alkyl alcohol mixture in the preparation of dialkyl carbonate by catalysed transesterification of a cyclic alkylene carbonate (e.g. ethylene carbonate or propylene carbonate) with alkyl alcohols. For an increase in the product quality of the dialkyl carbonate, the selection of the operating parameters of the dialkyl carbonate purification column are crucial, in order to reduce the formation of unwanted by-products such as alkoxy alcohols and aliphatic carbonate ethers.

Alkoxy alcohol forms from the reaction of alkylene oxide with the alkyl alcohol.

The aliphatic carbonate ether forms from the reaction of the alkoxy alcohol with dialkyl carbonate.

This carbonate ether, which generally has a higher boiling point than the alkyl alcohol, remains in the dialkyl carbonate. When the dialkyl carbonate is reacted with an aromatic monohydroxyl compound in a further process stage to give a diaryl carbonate, the aliphatic carbonate ether reacts further to give an aromatic carbonate ether. In the subsequent reaction of the diaryl carbonate with an aromatic dihydroxyl compound to give an aromatic polycarbonate, the aromatic carbonate ether leads to a deterioration in the product properties of the polycarbonate, with adverse effects both on the molecular weight, which, assuming the same reaction conditions, is lower in the presence of the aromatic carbonate ether than in its absence, and on the colour of the polymer.

The preparation of dialkyl carbonates from cyclic alkylene carbonate and alkyl alcohol, in which alkylene glycol is formed simultaneously as a by-product, is known and has been described many times. U.S. Pat. No. 6,930,195 B2 describes this catalysed transesterification reaction as a two-stage equilibrium reaction. In the first reaction stage, the cyclic alkylene carbonate reacts with alcohol to give hydroxyalkyl carbonate as an intermediate. The intermediate is then converted with the aid of alcohol in the second reaction stage to the products: dialkyl carbonate and alkylene glycol.

For the industrial implementation of the dialkyl carbonate preparation process, the use of a reactive distillation column (also referred to hereinafter as transesterification column), which has already been described inter alia in EP 530 615 A1, EP 569 812 A1 and EP 1 086 940 A1, has been found to be particularly favourable. In EP 569 812 A1, the cyclic alkylene carbonate is introduced continuously into the upper part of the transesterification column, and the alkyl alcohol comprising dialkyl carbonate into the middle or lower section of the transesterification column. In addition, below the introduction of the alkyl alcohol comprising dialkyl carbonate, virtually pure alkyl alcohol is introduced. A substance is referred to as virtually pure in the context of this invention when it comprises less than 2% by weight, preferably less than 1% by weight, of impurities. The high boiler mixture which includes the alkylene glycol prepared as a by-product is drawn off continuously at the bottom of the transesterification column. The low boiler mixture, which includes the dialkyl carbonate prepared, is drawn off at the top of the transesterification column as a dialkyl carbonate-alkyl alcohol mixture and subjected to a further purification step.

The distillation column for the purification of the dialkyl carbonate-alkyl alcohol mixture is operated at a higher pressure, such that a further dialkyl carbonate-alkyl alcohol mixture with a lower dialkyl carbonate content can be drawn off at the top of the column. The dialkyl carbonate as the main product is obtained at the bottom of this purification column.

For the development of an economically attractive preparation process for dialkyl carbonates, many factors play an important role. Most of the literature sources are concerned with the reaction parameters, for example conversion, selectivity, product purity or energy efficiency of the process (e.g. EP 1 760 059 A1, EP 1 967 242 A1 and EP 1 967 508 A1). Less commonly, the influencing factors for the formation of by-products in the dialkyl carbonate purification column are examined, even though these factors contribute to a not inconsiderable degree to the economic attractiveness of the process. Therefore, in this invention, measures are introduced for lowering by-product formation in the dialkyl carbonate purifying column.

EP 1 760 059 A1 describes a process for preparing dialkyl carbonate and alkylene glycol from alkylene carbonate and alkyl alcohol using a homogeneous catalyst. The reaction takes place in a distillation column (transesterification column). At the top of the column, a mixture consisting of dialkyl carbonate and alkyl alcohol is withdrawn and sent to a distillation column for separation of this mixture (dialkyl carbonate purifying column). In the bottom of this column, purified dialkyl carbonate is again drawn off. This dialkyl carbonate comprises an aliphatic carbonate ether which depends on the concentration of alkylene oxide in the alkylene carbonate which is supplied to the transesterification column. The alkylene carbonate was prepared by the reaction of alkylene oxide with carbon dioxide. At the end of the process for preparing alkylene carbonate, it is found that the alkylene carbonate still comprises small amounts of alkylene oxide. In the process for preparing dialkyl carbonate and alkylene glycol, it is found that the more alkylene oxide is present in the alkylene carbonate, the greater the concentration of the aliphatic carbonate ether in the purified dialkyl carbonate.

It is found that the concentration of the aliphatic carbonate ether in the purified dialkyl carbonate can be reduced only by the reduction of the content of alkylene oxide in the alkylene carbonate, which has to be ensured by means of complex apparatus in the preparation of the alkylene carbonate, for example by the use of a postreactor or of an additional distillation.

There was accordingly a need for a process for purifying dialkyl carbonate which is suitable for reducing the content of aliphatic carbonate ether with the same purity of the dialkyl carbonate, without additional apparatus complexity.

### BRIEF DESCRIPTION OF PREFERRED EMBODIMENTS

An embodiment of the present invention provides a process comprising purifying dialkyl carbonates of the formula (II) in one or more columns in the presence of an alkylene oxide of the formula (V) and of an alkyl alcohol of the formula (IV)

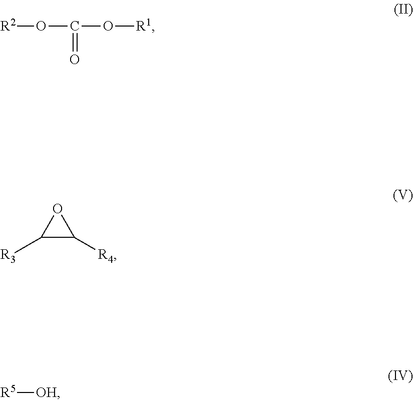

wherein
R¹ and R² represent, independently of one another, linear or branched, substituted or unsubstituted C₁-C₆-alkyl,
R³ and R⁴ represent, independently of one another, hydrogen, substituted or unsubstituted C₁-C₄-alkyl, substituted or unsubstituted C₂-C₄-alkenyl or substituted or unsubstituted C₆-C₁₂-aryl, and
R⁵ represents a straight-chain or branched C₁-C₄-alkyl; and
wherein the mean residence time of the dialkyl carbonate in the column(s) is from 0.3 h to 3 h.

### BRIEF DESCRIPTION OF THE SEVERAL VIEWS OF THE DRAWING

The foregoing summary, as well as the following detailed description of the invention, may be better understood when read in conjunction with the appended drawings. For the purpose of assisting in the explanation of the invention, there are shown in the drawings representative embodiments which are considered illustrative. It should be understood, however, that the invention is not limited in any manner to the precise arrangements and instrumentalities shown.

In the drawings:

FIG. 1 illustrates a process for purifying dialkyl carbonates an embodiment of the present invention.

FIG. 2 illustrates a process for purifying dialkyl carbonates according to another embodiment of the present invention.

FIG. 3 illustrates a process for purifying dialkyl carbonates according to another embodiment of the present invention.

FIG. 4 illustrates a process for purifying dialkyl carbonates according to another embodiment of the present invention.

FIG. 5 illustrates a process for purifying dialkyl carbonates according to another embodiment of the present invention.

### DETAILED DESCRIPTION OF PREFERRED EMBODIMENTS

As used herein, the singular terms “a” and “the” are synonymous and used interchangeably with “one or more” and “at least one,” unless the language and/or context cleary indicates otherwise. Accordingly, for example, reference to “a column” herein or in the appended claims can refer to a single column or more than one column. Additionally, all numerical values, unless otherwise specifically noted, are understood to be modified by the word “about.”

The present invention may therefore provide a process for purifying dialkyl carbonates, which leads to a lower content of aliphatic carbonate ether in the purified dialkyl carbonate compared to known processes.

It has now been found that, surprisingly, the content of by-products in the purified dialkyl carbonate, especially alkoxy alcohols and aliphatic carbonate ethers, can be reduced by a suitable selection of the range of the mean residence time of the substances in the dialkyl carbonate purifying column(s).

The mean residence time tᵣₜ of the liquid phase in the dialkyl carbonate purifying column(s) is preferably 0.3 to 3 h and more preferably 0.5 to 2 h. The mean residence time tᵣₜ is defined by the formula (I):

tᵣₜ:=V·ρ/(dm/dt_{DAC}) (I)
where:
V:=volume of the liquid holdup of distillation column below the feed point of the dialkyl carbonate-alcohol mixture
ρ:=mean density of the liquid holdup of distillation column below the feed point of the dialkyl carbonate-alcohol mixture
dm/dt_{DAC}:=mass flow withdrawn from the bottom of the distillation column, comprising the purified dialkyl carbonate.

Dialkyl carbonates purified in the context of the invention are preferably those of the general formula (II)

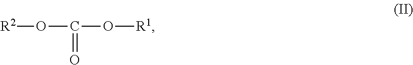

where R¹ and R² are each independently linear or branched, substituted or unsubstituted C₁-C₆-alkyl, preferably C₁-C₄-alkyl. R¹ and R² may be the same or different. R¹ and R² are preferably the same.

C₁-C₄-Alkyl in the context of the invention is, for example, methyl, ethyl, n-propyl, isopropyl, n-butyl, sec-butyl, tert-butyl, and C₁-C₆-alkyl is additionally, for example, n-pentyl, 1-methylbutyl, 2-methylbutyl, 3-methylbutyl, neopentyl, 1-ethylpropyl, cyclohexyl, cyclopentyl, n-hexyl, 1,1-dimethylpropyl, 1,2-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl or 1-ethyl-2-methylpropyl.

The above lists should be understood by way of example and not as a limitation.

Preferred dialkyl carbonates are dimethyl carbonate, diethyl carbonate, di(n-propyl) carbonate, di(iso-propyl) carbonate, di(n-butyl) carbonate, di(sec-butyl) carbonate, di(tert-butyl) carbonate or dihexyl carbonate. Particular preference is given to dimethyl carbonate or diethyl carbonate. Very particular preference is given to dimethyl carbonate.

The dialkyl carbonates are preferably prepared from cyclic alkylene carbonates with the formula (III):

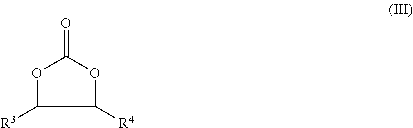

where R³ and R⁴ in the formula are each independently hydrogen, substituted or unsubstituted C₁-C₄-alkyl, substituted or unsubstituted C₂-C₄-alkenyl or substituted or unsubstituted C₆-C₁₂-aryl and R³ and R⁴ together with the two three-membered ring carbon atoms may be a saturated carbocyclic ring having 5-8 ring members.

Preferred alkylene carbonates are ethylene carbonate and propylene carbonate.

The cyclic alkylene carbonates are reacted with alcohols of the formula (IV)

R⁵—OH (IV)

where R⁵ is a straight-chain or branched C₁-C₄-alkyl.

Preferred alcohols are methanol and ethanol.

Alkylene oxides in the context of the process are compounds of the formula (V)

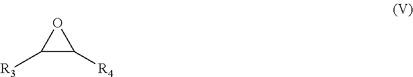

where R³ and R⁴ are each as defined above.

The distillation column for purifying the dialkyl carbonate preferably has a rectifying section having preferably 5 to 40 theoretical plates for concentration of the alkyl alcohol, and a stripping section having preferably 5 to 40 theoretical plates for concentration of the dialkyl carbonate.

In the column sections, in all parts of the dialkyl carbonate purifying column, i.e. both in the rectifying section and any stripping section, it is possible to use random packings or structured packings. The random packings or structured packings to be used are those customary for distillations, as described, for example, in Ullmann's Encyclopädie der Technischen Chemie, 4th ed., vol. 2, p. 528 ff. Examples of random packings include Raschig, Pall and Navolox rings, Interpack bodies, Berl, Intalex or Torus saddles. Examples of structured packings are sheet metal and fabric packings (for example BX packings, Montz Pak, Mellapak, Melladur, Kerapak and CY packing). The random packings and/or structured packings used can be produced from different materials, for example glass, stoneware, porcelain, stainless steel, plastic.

A suitable alternative is also column trays which are customary for distillations and are know to those skilled in the art; as specified, for example, in Henry Z. Kister, “Distillation—Design”, p. 259 ff. Examples of column trays include sieve trays, bubble-cap trays, valve trays and tunnel-cap trays.

Preference is given to random packings and structured packings which have a large surface area, good wetting and a sufficient residence time of liquid phase. These are, for example, Pall and Novolax rings, Berl saddles, BX packings, Montz Pak, Mellapak, Melladur, Rombopak, Kerapak and CY packings. The exact design of the stripping section to be used and of the rectifying section can be undertaken by the person skilled in the art.

The dimensions of the column bottom are in accordance with general rules known to those skilled in the art. As an alternative to a standard design of the column bottom, configuration measures can be effected to reduce the liquid content. For example, a constriction of the bottom diameter compared to the column body can be implemented, or a suitable baffle device for improving the degassing operation of the liquid in the column bottom can be installed and hence a lower residence time of the liquid can be established. In addition, the implementation of a suitable forced circulation evaporator system can adjust the residence time of the liquid in the column bottom. In addition, further measures are conceivable for reducing the liquid contents in the column bottom, for example the introduction of suitable displacement bodies.

The dialkyl carbonate and the alkyl alcohol are separated, preferably by distillation, in one or more distillation columns or in a combination of distillation and membrane separation—referred to hereinafter as a hybrid process (see, for example, U.S. Pat. No. 4,162,200 A, EP 581 115 B1, EP 592 883 B1 and WO 2007/096343A1).

If alkyl alcohol and dialkyl carbonate form an azeotrope (e.g. methanol and dimethyl carbonate), it is also possible to use a two-stage process, for example a two-pressure process, an extractive distillation, a heteroazeotropic distillation with a low-boiling entraining agent or a hybrid process. Particular preference is given to employing the two-pressure process or a hybrid process.

Very particular preference is given to performing the separation of the dialkyl carbonate and of the alkyl alcohol—even in the case that the dialkyl carbonate and the alkyl alcohol form an azeotrope—in a single distillation column. This distillation column is operated at a pressure which is higher than the pressure of the transesterification column(s). The operating pressure of the distillation column is in the range from 1 to 50 bar, preferably from 2 to 20 bar. At the bottom of the distillation column, the virtually pure dialkyl carbonate is withdrawn and, at the top, a mixture of dialkyl carbonate and alkyl alcohol. This mixture is supplied completely or partially to the transesterification column(s). If the process for preparing dialkyl carbonate is coupled to a process for preparing diaryl carbonate which is formed by transesterification of this dialkyl carbonate with an aromatic hydroxyl compound, a portion of the mixture of dialkyl carbonate and alkyl alcohol which is withdrawn at the top of the distillation column can be sent to an appropriate workup step for alkyl alcohol and dialkyl carbonate in the process stage for preparation of diaryl carbonate.

In a particularly preferred version, when the dialkyl carbonate and the alkyl alcohol form an azeotrope, this workup step is a two-pressure process. Such processes are known in principle to those skilled in the art (cf., for example, Ullmann's Encyclopedia of Industrial Chemistry, Vol. 7, 2007, Ch. 6.4. and 6.5.; Chemie Ingenieur Technik (67) November/1995).

If alkyl alcohol and dialkyl carbonate form an azeotrope, the distillate of a first distillation column of the process step for separation of dialkyl carbonate and alkyl alcohol preferably has a virtually azeotropic composition. In this case, the latter is preferably supplied in a two-pressure process to at least one further distillation column which works at an operating pressure below that of the first distillation column. As a result of the different operating pressure, the position of the azeotrope shifts to lower proportions of alkyl alcohol. The bottom product obtained in the second or further distillation column(s) is alkyl alcohol in a purity of 90 to 100% by weight, based on the total weight of the isolated bottom product, and the distillate obtained is a virtually azeotropic mixture. The second or further distillation column(s) which work(s) at lower operating pressure is/are, in very particularly preferred embodiments, preferably operated with the heat of condensation of the top condenser(s) of the first distillation column.

The two-pressure process exploits the pressure dependence of the azeotropic composition of a two-substance mixture. In the case of a mixture of alkyl alcohol and dialkyl carbonate, for example methanol and a methyl carbonate, the azeotropic composition shifts with increasing pressure to higher alkyl alcohol contents. If a mixture of these two components is supplied to a column (dialkyl carbonate column), and the alkyl alcohol content is below the corresponding azeotropic composition for the operating pressure of this column, the distillate obtained is a mixture with virtually azeotropic composition, and the bottom product obtained is virtually pure diallyl carbonate. The azeotropic mixture thus obtained is sent to a further distillation column (alkyl alcohol column). This works at a lower operating pressure compared to the dialkyl carbonate column. As a result, the position of the azeotrope is shifted to lower alkyl alcohol contents. As a result, it is possible to separate the azeotropic mixture obtained in the dialkyl carbonate column into a distillate with virtually azeotropic composition and virtually pure alkyl alcohol. The distillate of the alkyl alcohol column is fed back to the dialkyl carbonate column at a suitable point.

The operating pressure of the alkyl alcohol column is preferably selected such that it can be operated with the waste heat of the dialkyl carbonate column. The operating pressure is from 0.1 to 1 bar, preferably from 0.3 to 1 bar. The operating pressure of the dialkyl carbonate column is in the range from 1 to 50 bar, preferably from 2 to 20 bar.

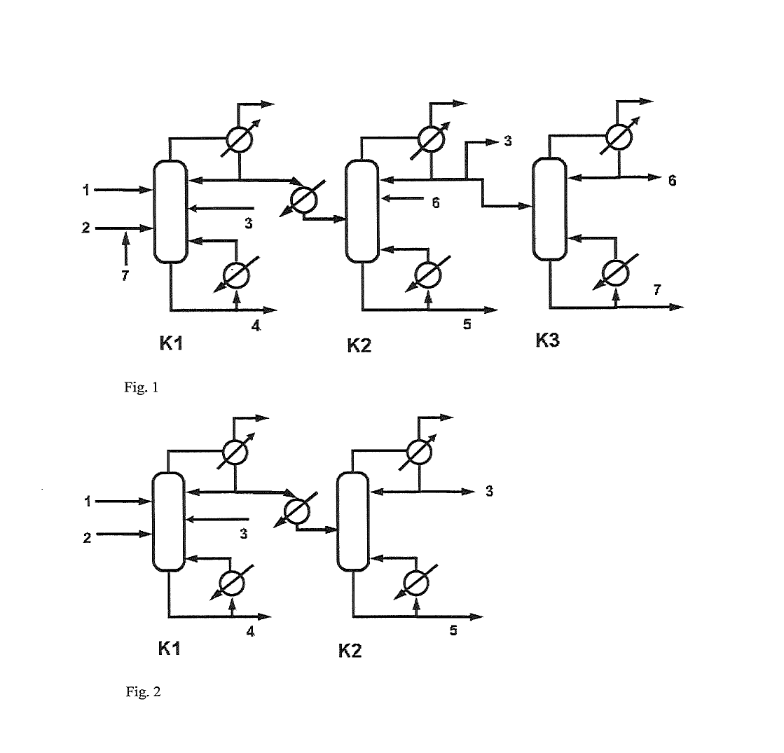

An illustrative reaction regime in the separation of dialkyl carbonate and alkyl alcohol by the two-pressure process is shown in FIG. 1.

A further preferred process for separating azeotropes of alkyl alcohol and dialkyl carbonate is the hybrid process. In the hybrid process, a two-substance mixture is separated by means of a combination of distillation and membrane process. This exploits the fact that the components can be separated at least partially from one another by means of membranes due to their polar properties and their different molecular weights. In the case of a mixture of alkyl alcohol and dialkyl carbonate, for example methanol and dimethyl carbonate, in the case of use of suitable membranes, pervaporation or vapour permeation affords an alkyl alcohol-rich mixture as permeate and an alkyl alcohol-depleted mixture as retentate. If a mixture of these two components is fed to a column (dialkyl carbonate column), the alkyl alcohol content being below the corresponding azeotropic composition for the operating pressure of this column, the distillate obtained is a mixture with a distinctly increased alkyl alcohol content compared to the feed, and the bottom product obtained is virtually pure dialkyl carbonate.

In the case of a hybrid process composed of distillation and vapour permeation, the distillate of the column is withdrawn in vaporous form. The vaporous mixture thus obtained is supplied to a vapour permeation, optionally after superheating. The vapour permeation is operated by establishing virtually the operating pressure of the column on the retentate side, and a lower pressure on the permeate side. The operating pressure of the column is in the range from 1 to 50 bar, preferably from 1 to 20 bar and more preferably from 2 to 10 bar. The pressure on the permeate side is from 0.05 to 2 bar. This affords, on the permeate side, an alkyl alcohol-rich fraction with an alkyl alcohol content of at least 70% by weight, preferably at least 90% by weight, based on the total weight of the fraction. The retentate, which comprises a reduced alcohol content compared to the distillate of the column, is optionally condensed and fed back to the distillation column.

In the case of a hybrid process composed of distillation and pervaporation, the distillate of the column is withdrawn in liquid form. The mixture thus obtained is supplied to a pervaporation, optionally after heating. The pervaporation is operated by establishing an identical or increased operating pressure compared to the column on the retentate side, and a lower pressure on the permeate side. The operating pressure of the column is in the range from 1 to 50 bar, preferably from 1 to 20 bar and more preferably from 2 to 10 bar. The pressure on the permeate side is from 0.05 to 2 bar. On the permeate side, an alkyl alcohol-rich vaporous fraction is obtained with an alkyl alcohol content of at least 70% by weight, preferably at least 90% by weight, based on the total weight of the fraction. The liquid retentate, which obtains a reduced alkyl alcohol content compared to the distillate of the column, is fed back to the distillation column. As a result of the evaporation of the permeate, heat is required, which may not be present to a sufficient degree in the feed stream for pervaporation. Therefore, a membrane separation by means of pervaporation can optionally be heated with additional heat exchangers, in which case they are integrated or optionally installed between several pervaporation steps connected in series.

In the case of a hybrid process, the separation of dialkyl carbonate and alkyl alcohol is more preferably effected by means of a combination of distillation and vapour permeation.

The heat required for separation of alkyl alcohol and dialkyl carbonate is supplied at a temperature of 100° C. to 300° C., preferably of 100° C. to 230° C., and more preferably of 120° C. to 210° C., especially more preferably of 140° C. to 190° C.

The process for preparing the dialkyl carbonate can be performed continuously or batchwise. Preference is given to continuous mode.

In the process, the cyclic alkylene carbonate compound(s) and the alcohol(s) are used preferably in a molar ratio of 1:0.1 to 1:40, more preferably of 1:1.0 to 1:30, most preferably of 1:2.0 to 1:20. The molar ratio stated does not take account of the recycling of cyclic alkylene carbonate compound or alcohol into the transesterification column via one or more top condenser(s) (cf. under (b)) or one or more bottom evaporator(s) which may be present.

The catalyst is preferably introduced into the column together with the stream comprising the cyclic alkylene carbonate in dissolved or suspended form into the transesterification column via an introduction point which is arranged above the introduction points for the alcohol. Alternatively, the catalyst can also be metered in separately, for example, dissolved in the alcohol, in the alkylene glycol or in a suitable inert solvent. In the case of use of heterogeneous catalysts, they can be used in a mixture with the random packings mentioned, in a suitable form in place of random packings, or as a bed on any column trays incorporated.

The process for preparing dialkyl carbonate is performed in a transesterification column. In preferred embodiments of the preparation process, the liquid stream withdrawn at the bottom of this transesterification column—optionally after concentration—can be subjected to a further reaction and/or purification in one or more further steps. Preferably, individual steps or all such further steps can be effected in one or more further columns.

Useful transesterification columns or optionally second or further column(s) may be columns known to those skilled in the art. These are, for example, distillation or rectification columns, preferably reactive distillation or reactive rectification columns.

The transesterification column comprises preferably at least one rectifying section in the upper part of the column and at least one reaction zone below the rectifying section. The rectifying section has preferably 0 to 30, preferably 0.1 to 30, theoretical plates.

In preferred embodiments, the transesterification column has at least one stripping section below a reaction zone.

The transesterification column may additionally preferably be equipped with one or more bottom evaporator(s). In the case of execution of the transesterification column with a stripping section, preference is given to additionally using a bottom evaporator which fully or partly evaporates the liquid effluxing from the stripping section. This fully or partly evaporated liquid stream is recycled fully or partly back into the transesterification column. In the case of an embodiment without a stripping section, in any bottom evaporator used, the liquid effluxing out of the reaction zone is fully or partly evaporated and fully or partly recycled back into the transesterification column.

The rectifying section(s) may, in preferred embodiments, be accommodated in the transesterification column together with the reaction section(s) and optionally at least one stripping section. In this case, the vaporous mixture coming from the reaction zone(s) is passed from below into a lower section of the rectifying section, or if appropriate to the lower rectifying section, and depletion of the alkylene carbonate or alkylene glycol takes place.

Below the reaction zone and any stripping section present, a mixture comprising alkylene glycol, excess or unconverted alkylene carbonate, alcohol, dialkyl carbonate, transesterification catalysts and high-boiling compounds which have formed in the reaction or were already present in the reactants is obtained. In the case of use of a stripping section, the content of low-boiling compounds, for example dialkyl carbonate and alcohol, is reduced, though further dialkyl carbonate and alkylene glycol are formed under some circumstances in the presence of the transesterification catalyst. The energy required for this purpose is preferably supplied through one or more evaporators.

In all sections of the transesterification column, i.e. both in the rectifying section and any stripping section and in the reaction zone, random packings or structured packings can be used. The random packings or structured packings to be used are those customary for distillations, as described, for example, in Ullmann's Encyclopädie der Technischen Chemie, 4th ed., vol. 2, p. 528 ff. Examples of random packings include Raschig or Pall and Novalox rings, Berl, Intalex or Torus saddles, Interpack bodies, and examples of structured packings include sheet metal and fabric packings (for example BX packings, Montz Pak, Mellapak, Melladur, Kerapak and CY packing) made from various materials, such as glass, stoneware, porcelain, stainless steel, plastic. Preference is given to random packings and structured packings which have a large surface area, good wetting and sufficient residence time of the liquid phase. These are, for example, Pall and Novalox rings, Berl saddles, BX packings, Montz Pak, Mellapak, Melladur, Kerapak and CY packings.

Another suitable alternative is column trays, for example sieve trays, bubble-cap trays, valve trays, tunnel-cap trays. In the reaction zone(s) of the transesterification column, particular preference is given to column trays with high residence times and good mass transfer, for example bubble-cap trays, valve trays or tunnel-cap trays with high overflow weirs. The number of theoretical plates of the reaction zone is preferably 3 to 50, more preferably 10 to 50 and most preferably 10 to 40. The liquid holdup is preferably 1 to 80%, more preferably 5 to 70% and most preferably 7 to 60% of the internal column volume of the reaction zone. The exact design of the reaction zone(s), of any stripping section to be used and of the rectifying section(s) can be undertaken by the person skilled in the art.

The temperature of the reaction zone(s) is preferably in the range from 20 to 200° C., more preferably from 40 to 180° C., most preferably from 40 to 160° C. It is advantageous to perform the esterification not only at standard pressure but also at elevated or reduced pressure. The pressure of the reaction zone is therefore preferably in the range from 0.2 to 20 bar, more preferably from 0.3 to 10 bar, most preferably from 0.4 to 5 bar. The pressure figures above and given hereinafter are—unless explicitly mentioned otherwise—absolute pressure figures.

The vapour mixture comprising dialkyl carbonate and alkyl alcohol which is withdrawn at the top of the transesterification column in the process for preparing the dialkyl carbonate, after condensation at the top of the transesterification column, is preferably fed fully or partly to at least one further process step comprising at least one distillation column for separation of dialkyl carbonate and alkyl alcohol.

### EXPLANATIONS FOR THE FIGURES

K₁ transesterification column
K₂ first distillation column for separation of the mixture comprising dialkyl carbonate and alkyl alcohol
K₃ second distillation column for separation of the mixture comprising dialkyl carbonate and alkyl alcohol
   1 Reactant stream comprising alkylene carbonate and/or optionally catalysts
   2 Reactant stream comprising virtually pure alkyl alcohol
   3 Reactant stream comprising alkyl alcohol and dialkyl carbonate
   4 Stream comprising alkylene glycol
   5 Stream comprising purified dialkyl carbonate
   6 Stream comprising dialkyl carbonate and alkyl alcohol
   7 Stream comprising virtually pure alkyl alcohol
   8 Stream comprising extractant (preferably alkylene carbonate)
   9 Stream comprising extractant (preferably alkylene carbonate)
   10 Stream comprising extractant (preferably alkylene carbonate)

FIG. 1 describes a transesterification step of alkylene carbonate and alkyl alcohol by means of reactive rectification in a first transesterification column (K1) in general, and the workup of the mixture comprising dialkyl carbonate and alkyl alcohol obtained at the top of the transesterification column by means of two-pressure distillation in a first (K2) and a second (K3) distillation column.

FIG. 2 describes a transesterification step of alkylene carbonate and alkyl alcohol by means of reactive rectification in a first transesterification column (K1) in general, and the workup of the mixture comprising dialkyl carbonate and alkyl alcohol obtained at the top of the transesterification column by means of a single distillation column (K2).

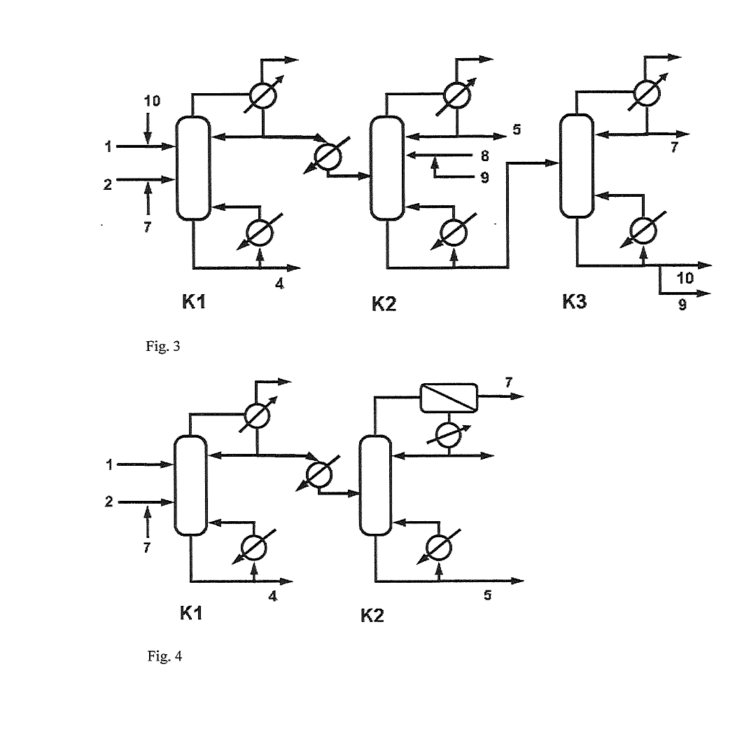

FIG. 3 describes a transesterification step of alkylene carbonate and alkyl alcohol by means of reactive rectification in a first transesterification column (K1) in general, and the workup of the mixture comprising dialkyl carbonate and alkyl alcohol obtained at the top of the transesterification column by means of extractive distillation in a first (K2) and a second (K3) distillation column, preference being given to using the alkylene carbonate as the extractant.

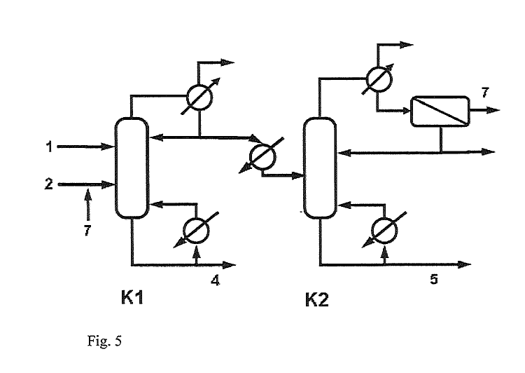

FIG. 4 describes a transesterification step of alkylene carbonate and alkyl alcohol by means of reactive rectification in a first transesterification column (K1) in general, and the workup of the mixture comprising dialkyl carbonate and alkyl alcohol obtained at the top of the transesterification column by means of distillation and vapour permeation in a distillation column (K2).

FIG. 5 describes a transesterification step of alkylene carbonate and alkyl alcohol by means of reactive rectification in a first transesterification column (K1) in general, and the workup of the mixture comprising dialkyl carbonate and alkyl alcohol obtained at the top of the transesterification column by means of distillation and pervaporation in a distillation column (K2).

The examples which follow serve to illustrate the invention by way of example and should not be interpreted as a restriction.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

All the references described above are incorporated by reference in their entireties for all useful purposes.

### EXAMPLES

An example is now used to show the preferred mode of operation for the process according to the invention in detail. Example 1 shows the preferred mode of operation for the dialkyl carbonate purifying column. This example should in no way be interpreted as a limitation of the invention.

The advantage of the process according to the invention, namely the reduction of the formation of unwanted by-products such as alkoxy alcohols and aliphatic carbonate ethers by the suitable establishment of the residence time of the reactants in the liquid phase within the purifying column is shown hereinafter with the aid of two comparative examples.

Both in example 1 and in the comparative examples, dimethyl carbonate as the dialkyl carbonate and ethylene glycol form from the reaction between ethylene carbonate and methanol. Methoxyethanol in this case is the alkoxy alcohol, and methyl methoxyethyl carbonate (MMEC) is the aliphatic carbonate ether.

### Example 1

A reactive distillation column consists of a rectifying section with 9 theoretical plates, a reaction zone with 25 reaction trays (holdup/tray: 0.6 m³) and a stripping section with 4 theoretical plates. The column is operated at a pressure of 400 mbar (absolute) at the top of a column and a mass-based return ratio of 0.585.

In the upper column region, directly above the first reaction tray, 9000 kg/h of ethylene carbonate with an ethylene oxide content of 100 ppm and 174 kg/h of a mixture containing 33.3% by weight of KOH and 66.7% by weight of ethylene glycol are metered in continuously. Between the 8th and 9th reaction trays, the returned distillate stream of the dialkyl carbonate purifying column is fed in in vaporous form with a mass flow of 21371 kg/h. In addition, at the lower end of the reaction zone, 7124 kg/h of a vapour mixture comprising 99.5% by weight of methanol, 0.4% by weight of ethylene glycol and small amounts of dimethyl carbonate and other substances are fed in.

The bottom evaporator is operated at 102° C., and 7018 kg/h of liquid bottom product comprising principally ethylene glycol are obtained.

A partial condenser condenses the vapour stream at the top of the column at 40° C. As a result, 6 kg/h of vaporous distillate are drawn off. The liquid distillate with a mass flow of 30645 kg/h is fed to a further distillation column for further purification.

The distillation column for purification of the dialkyl carbonate which forms in the transesterification, consisting of a rectifying section with 28 theoretical plates and a stripping section with 11 theoretical plates, is operated at a pressure of 10 bar (absolute) at the top of the column and a mass-based return ratio of 1.0.

In the lower region of the column, between the 27th and 28th theoretical plates, 30645 kg/h of a dialkyl carbonate-containing alcohol mixture comprising 59% by weight of methanol and 41% by weight of dimethyl carbonate are metered in continuously.

A partial condenser condenses the vapour stream at the top of the column at 137° C. This affords both 21 kg/h of vaporous distillate with a composition of 82.9% by weight of methanol, 14.4% by weight of dimethyl carbonate, 0.3% by weight of ethylene oxide and 2.4% by weight of CO₂, and 21380 kg/h of liquid distillate with a composition of 84% by weight of methanol and 16% by weight of dimethyl carbonate. To avoid enrichment of low-boiling components, a purge stream of 9 kg/h is withdrawn from the distillate stream and 21371 kg/h are recycled to the transesterification column.

The 1st to 39th stages each have a liquid holdup of 0.06 m³. The bottom of the column has a liquid holdup of 16.5 m³. The temperature of liquid in the bottom of the column is 183° C. The mean density of liquid holdup is 860 kg/m³. The mean residence time is 1.6 h.

This affords 9244 kg/h of liquid bottom product comprising 99.5% by weight of dimethyl carbonate. In addition to methanol, 11 ppm of methoxy ethanol and 5 ppm of MMEC are present.

### Comparative Example 1

The same construction of the columns as described in Example 1 is used. The column for purification of the dialkyl carbonate is operated at a pressure of 10 bar (absolute) at the top of the column and a mass-based return ratio of 1.0.

In the lower region of the column for purifying the dialkyl carbonate, between the 27th and 28th theoretical plates, 30645 kg/h of a dialkyl carbonate-containing alcohol mixture comprising 59% by weight of MeOH and 41% by weight of dimethyl carbonate are metered in continuously.

A partial condenser condenses the vapour stream at the top of the column at 137° C. This affords both 21 kg/h of vaporous distillate with a composition of 83.3% by weight of methanol, 14.6% by weight of dimethyl carbonate, 0.3% by weight of ethylene oxide and 1.8% by weight of CO₂, and 21380 kg/h of liquid distillate with a composition of 84% by weight of methanol and 16% by weight of dimethyl carbonate. To avoid enrichment of low-boiling components, a purge stream of 9 kg/h is withdrawn from the distillate stream and 21371 kg/h are recycled to the transesterification column.

The 1st to 39th stages each have a liquid holdup of 0.3 m³. The bottom of the column has a liquid holdup of 25 m³. The temperature of liquid in the bottom of the column is 183° C. The mean density of liquid holdup is 860 kg/m³. The mean residence time is 2.6 h.

This affords 9244 kg/h of liquid bottom product comprising 99.5% by weight of dimethyl carbonate. In addition to methanol, 38 ppm of methoxy ethanol and 24 ppm of MMEC are present.

### Comparative Example 2

The same construction of the columns as described in Example 1 is used. The column for purification of the dialkyl carbonate is operated at a pressure of 20 bar (absolute) at the top of the column and a mass-based return ratio of 1.0.

The increase in the operating pressure of the dialkyl carbonate purifying column and the pressure dependence of the composition of the methanol/dimethyl carbonate azeotrope lead to altered operating conditions in the transesterification column, which are detailed below.

The transesterification column is operated at a pressure of 400 mbar (absolute) at the top of the column and a mass-based reflux ratio of 0.585. In the upper region of the column, directly above the first reaction tray, 9000 kg/h of ethylene carbonate with an ethylene oxide content of 100 ppm and 174 kg/h of a mixture comprising 33.3% by weight of KOH and 66.7% by weight of ethylene glycol are metered in continuously. Between the 8th and 9th reaction trays, the recycled distillate stream of the dialkyl carbonate purifying column is fed only in vaporous form with a mass flow of 21371 kg/h and a composition of 90.5% by weight of methanol and 9.5% by weight of dimethyl carbonate. In addition, 7124 kg/h of a vapour mixture comprising 99.5% by weight of methanol, 0.4% by weight of ethylene glycol and small amounts of dimethyl carbonate and other substances are supplied at the lower end of the reaction zone. The bottom evaporator is operated at 102° C., and 7018 kg/h of liquid bottom product comprising principally ethylene glycol are obtained. A partial condenser condenses the vapour stream at the top of the column at 40° C. As a result, 6 kg/h of vaporous distillate are drawn off. The liquid distillate with a mass flow of 30645 kg/h is supplied to the dialkyl carbonate purifying column for further purification.

Analogously to Example 1 and Comparative Example 1, the distillation column for purifying the dialkyl carbonate formed in the transesterification consists of a rectifying section with 28 theoretical plates and a stripping section with 11 theoretical plates. The purifying column is operated at a pressure of 20 bar (absolute) at the top of the column and a mass-based return ratio of 1.0.

In the lower region of the column for purifying the dialkyl carbonate, between the 27th and 28th theoretical plates, 30645 kg/h of a dialkyl carbonate-containing alcohol mixture comprising 63.4% by weight of MeOH and 36.6% by weight of dimethyl carbonate are metered in continuously.

A partial condenser condenses the vapour stream at the top of the column at 167° C. This affords both 21 kg/h of vaporous distillate with a composition of 91.4% by weight of methanol, 7.7% by weight of dimethyl carbonate, 0.7% by weight of ethylene oxide and 0.2% by weight of CO₂, and 21374 kg/h of liquid distillate with a composition of 90.5% by weight of methanol and 9.5% by weight of dimethyl carbonate. To avoid enrichment of low-boiling components, a purge stream of 3 kg/h is withdrawn from the distillate stream and 21371 kg/h are recycled to the transesterification column.

The 1st to 39th stages each have a liquid holdup of 0.3 m³. The bottom of the column has a liquid holdup of 25 m³. The temperature of liquid in the bottom of the column is 224° C. The mean density of liquid holdup is 750 kg/m³. The mean residence time is 2.3 h.

This affords 9250 kg/h of liquid bottom product comprising 99.5% by weight of dimethyl carbonate. In addition to methanol, 53 ppm of methoxy ethanol and 111 ppm of MMEC are present.

1. A process comprising purifying dialkyl carbonates of the formula (II) in one or more columns in the presence of an alkylene oxide of the formula (V) and of an alkyl alcohol of the formula (IV) 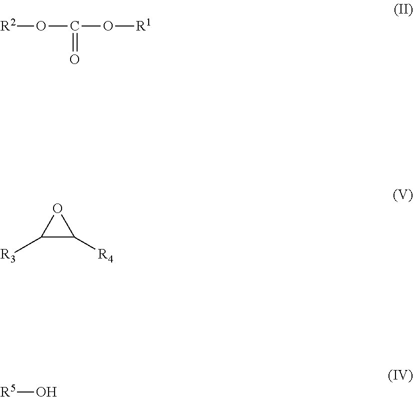
wherein R¹ and R² represent, independently of one another, linear or branched, substituted or unsubstituted C₂-C₆-alkyl,
R³ and R⁴ represent, independently of one another, hydrogen, substituted or unsubstituted C₁-C₄-alkyl, substituted or unsubstituted C₂-C₄-alkenyl or substituted or unsubstituted C₆-C₁₂-aryl, and
R⁵ represents a straight-chain or branched C₁-C₄-alkyl; and
wherein the mean residence time of the dialkyl carbonate in the column(s) is from 0.3 h to 3 h.

2. The process according to claim 1, wherein the mean residence time of the dialkyl carbonate in the column(s) is from 0.5 h to 2 h.

3. The process according to claim 1, wherein the alcohol comprises methanol or ethanol.

4. The process according to claim 2, wherein the alcohol comprises methanol or ethanol.

5. The process according to any of claim 1, wherein the alkylene comprises ethylene or propylene.

6. The process according to any of claim 4, wherein the alkylene comprises ethylene or propylene.

7. The process according to claim 1, wherein the pressure in at least one of the columns used for purification of the dialkyl carbonate is from 0.5 to 50 bar absolute.

8. The process according to claim 6, wherein the pressure in at least one of the columns used for purification of the dialkyl carbonate is from 0.5 to 50 bar absolute.

9. The process according to claim 1, wherein the pressure in at least one of the columns used for purification of the dialkyl carbonate is from 2 to 20 bar absolute.

10. The process according to claim 8, wherein the pressure in at least one of the columns used for purification of the dialkyl carbonate is from 2 to 20 bar absolute.

11. The process according to claim 1, wherein the temperature in at least one of the columns used for purification of the dialkyl carbonate is from 120° C. to 210° C.

12. The process according to claim 10, wherein the temperature in at least one of the columns used for purification of the dialkyl carbonate is from 120° C. to 210° C.

13. The process according to claim 1, wherein at least one of the columns used for purification of the dialkyl carbonate is a packed column.

14. The process according to claim 12, wherein at least one of the columns used for purification of the dialkyl carbonate is a packed column.
